(19) United States
(12) Patent Application Publication
(10) Pub. No.: US 2004/0247775 A1
(43) Pub. Date: Dec. 9, 2004

(54) Positive displacement coating deposition apparatus and method

(76) Inventors: Dennis R. Boulais, Danielson, CT (US); Samuel J. Epstein, Newton, MA (US); Wendy Naimark, Cambridge, MA (US); Praveen Kulkarni, Worcester, MA (US)

Correspondence Address: KENYON & KENYON, 1500 K STREET, N.W., SUITE 700, WASHINGTON, DC, 20005, US

(21) Appl. No.: 10/455,315

(22) Filed: Jun. 6, 2003

(51) Int. Cl. A61L 2/00; B05D 3/00; B05C 11/00

(52) U.S. Cl. 427/2.1; 118/668

(57) ABSTRACT

The present invention provides an apparatus and method that uses positive displacement of coating material using a computer controlled, motorized dispensing device. The flow rate of the dispensing device is controlled, and the positive displacement apparatus and method result in a precise amount of coating that is dispensed. The positive displacement coating apparatus and method allow for much more accurate and consistent coating from part to part. Because the positive displacement coating apparatus precisely controls the flow rate of the coating, differences in viscosity of the coating do not adversely affect the amount of the coating that is dispensed. In addition, the fluid flow path or pressure differential do not adversely affect the amount of the coating that is dispensed.

### FIELD OF THE INVENTION

The present invention relates generally to an apparatus and method for applying coating to a work piece. In a specific application, the present invention relates to an apparatus and method for applying coating such as therapeutic materials or DNA on the surface of an implantable medical device such as a stent.

### BACKGROUND INFORMATION

The positioning and deployment of medical devices within a target site of a patient is a common, often-repeated procedure of contemporary medicine. Such devices are used for a variety of medical purposes.

Coatings are often applied to these medical devices to increase their effectiveness. These coatings may provide a number of benefits, including reducing the trauma suffered during the insertion procedure, facilitating the acceptance of the medical device into the target site, and improving the post-procedure effectiveness of the device.

Expandable stents, stent grafts, balloon delivery systems, and aneurism coils are specific examples of medical devices or implants that may be coated and inserted within the body. Expandable stents are tube-like medical devices that often have a mesh-like structure designed to support the inner walls of a lumen. These stents are typically positioned within a lumen and, then, expanded to provide internal support for it. Because of the direct contact of the stent with the inner walls of the lumen, stents have been coated with various compounds and therapeutics to enhance their effectiveness.

When a coating is applied to a stent haphazardly, or if the coating has somehow been removed during the stent's manufacture or delivery, the stent's effectiveness can be compromised.

Indiscriminate coating methods such as dip-coating and spray-coating have been used to coat stents as well as other medical devices. These methods are, however, both difficult to control and wasteful. For example, dipping can result in non-uniform application of the coating to the device, because gravity causes more coating to be applied at one end or region of the device. This makes it difficult to predict the dosage of therapeutic that will be delivered when the stent or other device is implanted. In the case of stents, the indiscriminate nature of dipping is also problematic as it may lead to the cracking and/or crumbling of coating at the junctions, hinges, and/or flexing members of the mesh-like stents. The coating that covers these portions of the stent is highly susceptible to becoming removed because, as the stent is expanded, intolerable stresses may develop within the coating. In addition, indiscriminate coating such as dip-coating and spray coating may lead to undesirable “webbing” of coating between stent members. Webbing of coating in the areas between stent members is unlikely to be held against the vessel wall, and this coating material may be lost during deployment.

Current coating methods like spray-coating are also wasteful because they result in large amounts of the coating being lost during the process. In the case of expensive agents to be coated such as DNA, such wasteful processes make the coating method prohibitive.

The assignee of the current patent application is also the assignee of other patent applications directed to resolving some or all of the problems noted above. These include U.S. patent application Ser. No. 09/895,415, filed Jul. 2, 2001, entitled “Coating a Medical Appliance with a Bubble Jet Printing Head,” and U.S. patent application Ser. No. 10/045,492, filed Jan. 14, 2002, entitled “Coating Dispensing System and Method Using a Solenoid Head for Coating Medical Devices.” The disclosures of these applications are hereby incorporated herein by reference.

Certain previously-proposed coating techniques have relied on pressurized containers to cause the dispensing of the coating. Because of this, the actual amount of coating that is dispensed is highly dependent upon the pressure in the fluid container, the viscosity of the fluid, and the internal shape of the fluid path in the dispensing device. This results in variations in the amount of the coating dispensed, making it difficult to reproduce the same coating results from part to part.

### SUMMARY OF THE INVENTION

An object of the invention is to provide a novel apparatus and method for applying coating to a work piece in an efficient and effective manner.

In accordance with the invention, in certain embodiments, the apparatus and method provide for precision control of the amount of coating that is applied at precise locations on the target device. For example, the apparatus and method in certain embodiments permit the application of precise amounts of coating directly to a stent surface. The apparatus and method may be used to dispense coating in a desired pattern, which may, if desired, follow the pattern of the stent surface.

In accordance with the invention, in certain embodiments, the apparatus and method are useful for applying expensive coatings, such as DNA coatings, because the apparatus and method reduce or eliminate waste of the coating material.

In accordance with the invention, in certain embodiments, the apparatus and method are useful for applying relatively viscous coatings. For example, in certain embodiments, an apparatus and method in accordance with the invention are suitable for handling coating materials that have a viscosity in excess of 40 centipoise. In certain embodiments, an apparatus and method in accordance with the invention are suitable for handling coating materials that have a viscosity in excess of 100 centipoise. An apparatus and method in accordance with certain embodiments can handle highly viscous coatings, such as DNA coatings or other highly viscous coatings among those described below.

The present invention provides an apparatus and method that use positive displacement of the coating material using a computer controlled, motorized dispensing device. The flow rate of the dispensing device is controlled, and the positive displacement apparatus and method result in a precise amount of coating that is dispensed. The positive displacement coating apparatus and method allow for much more accurate and consistent coating from part to part.

Because the positive displacement coating apparatus of the present invention precisely controls the flow rate of the coating, differences in viscosity of the coating do not adversely affect the amount of the coating that is dispensed. In addition, unlike some prior coating methods, the fluid flow path or pressure differential do not adversely affect the amount of the coating that is dispensed.

In accordance with certain embodiments, a positive displacement coating apparatus may be used as part of a system for applying a coating to medical devices having accessible patterned surfaces, for example stents. This system may include: a work piece holder, a vision system, and a positive displacement coating apparatus. In this system the work piece holder, or appliance support, may be adapted to hold the medical device and to provide direct access for a coating to contact the exposed external patterned surfaces of the medical device. The positive displacement coating apparatus in this system may move with respect to the medical device and may be in communication with a source of coating and with a computer processor. The processor in this system may contain commands that instruct the positive displacement coating apparatus to force coating onto accessible surfaces of the medical device in a predetermined pattern. That pattern may, if desired, correlate with the accessible patterned surface of the medical device.

A method for applying a coating to a medical device having an accessible surface is also provided. In one embodiment this method may include holding the medical device, providing direct access to an external surface of the medical device, and receiving command signals that instruct a positive displacement coating apparatus to force coating onto the accessible surface of the medical device. The coating may be dispensed in a pattern that correlates with the accessible patterned surface of the medical device.

### BRIEF DESCRIPTION OF THE DRAWINGS

FIG. 1A shows an embodiment of a positive displacement coating apparatus in accordance with the invention.

FIG. 1B shows an alternative arrangement for a positive displacement coating apparatus in accordance with the invention, incorporating a valve.

FIG. 2 is an enlarged view of a nozzle portion of a positive displacement coating apparatus in accordance with the invention, applying a coating to a portion of a stent.

FIG. 3 illustrates an alternative vane type embodiment for a mechanical dispenser portion of a positive displacement coating apparatus in accordance with the invention.

FIG. 4 illustrates an alternative bellows type embodiment for a mechanical dispenser portion of a positive displacement coating apparatus in accordance with the invention.

FIG. 5 illustrates an alternative bladder type embodiment for a mechanical dispenser portion of a positive displacement coating apparatus in accordance with the invention.

FIG. 6 illustrates an alternative screw type embodiment for a mechanical dispenser portion of a positive displacement coating apparatus in accordance with the invention.

FIG. 7 is a schematic view of a system for applying a coating to a medical device using a positive displacement coating apparatus in accordance with an embodiment of the invention.

### DETAILED DESCRIPTION

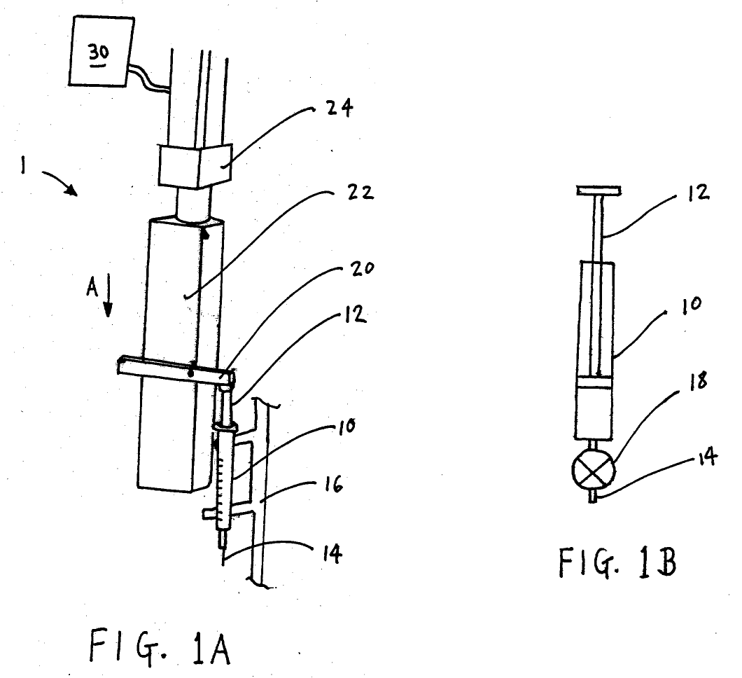

FIG. 1A illustrates an embodiment of a positive displacement coating apparatus 1 in accordance with the invention. The apparatus 1 in this embodiment comprises a piston type mechanical dispenser having a syringe barrel 10 and a syringe plunger 12. Attached at the outlet end of the syringe barrel 10 is a dispensing nozzle 14. The syringe barrel 10 may be mounted on a suitable stand or bracket 16.

The syringe plunger 12 is movable longitudinally within the syringe barrel 10. A pusher block 20 is mounted to press against the syringe plunger 12. The pusher block 12 is in turn connected to a linear actuator 22, which is actuated by a servo motor 24. The servo motor may be controlled by a suitable computer processor, shown schematically in FIG. 1A by block 30.

The computer processor 30 sends signals to the servo motor 24 to control its motion. When activated, the servo motor 24 actuates the linear actuator 22, causing it to move in the direction shown by arrow A. This in turn causes the pusher block 20 to move in the same direction, which forces the syringe plunger 12 downwardly into the syringe barrel 10.

The desired coating is located within the syringe barrel 10. When the syringe plunger 12 moves downwardly into the syringe barrel 10, it forces the coating out of the dispensing nozzle 14 and onto the desired work piece, for example a stent.

Because the syringe plunger 12 acts directly on the coating, and because the mechanical displacement of the syringe plunger 12 causes the dispensing of the coating, the flow rate of the coating out of the dispensing nozzle 14 can be controlled precisely. Controlling the rate of movement of the syringe plunger 12 controls the rate of flow of coating out of the dispensing nozzle 14.

FIG. 1B illustrates an alternative embodiment of a positive displacement coating apparatus in accordance with the invention. The apparatus in this embodiment also comprises a piston type mechanical dispenser having a syringe barrel 10, a syringe plunger 12, and a dispensing nozzle 14, similar to those in FIG. 1A. In addition, this embodiment includes a valve 18 that may be used to turn the flow on and off. In all other respects, the embodiment may be similar to that in FIG. 1A.

The incorporation of a valve 18 may be used when applying compressible fluids such as DNA and hydrogels, or those viscous fluids with entrapped air bubbles. Without a valve, when dispensing such fluids, flow may tend to continue after the syringe plunger 12 was stopped, because of the expansion of the fluid and/or bubbles. The valve 18, shown close to the end of the dispensing nozzle 14, may be used to turn off the flow, for example when the syringe plunger 12 is stopped. The syringe plunger 12 and valve 18 in this arrangement can be used to apply a constant pressure.

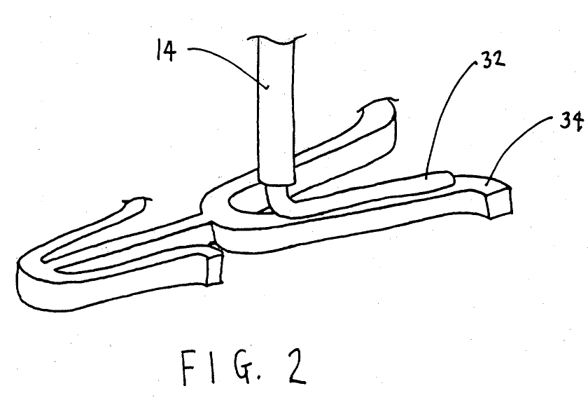

FIG. 2 shows an enlarged view of a portion of a dispensing nozzle 14 of a positive displacement coating apparatus in accordance with the invention. As illustrated, the positive displacement coating apparatus allows controlled dispensing of a coating 32. In this Figure, the coating 32 is being applied precisely along the external surface of a stent 34, a portion of which is illustrated.

To enable the coating 32 to be dispensed precisely along the pattern of the external surface of the stent 34, a computer processor may be used to control the movement of the stent 34 as the coating 32 is dispensed from the positive displacement coating apparatus. The control of the movement of stent 34 can be coordinated with the control of the dispensing of coating from dispensing nozzle 14. As an alternative to moving the stent 34, the dispensing nozzle 14 (and, if desired, other portions of the positive displacement coating apparatus) may be moved to follow the pattern of stent 34 as the coating 32 is dispensed. More generally, in certain embodiments, the medical device may be moved during coating while the dispensing nozzle is held in place, while in other embodiments, the dispensing nozzle may be moved during coating while the medical device is held in place. Also, both the dispensing nozzle and the medical device may continuously or intermittently be moved during coating. In addition, the stent and/or dispensing nozzle may be moved such that the location of the dispensing nozzle relative to the stent travels in a fixed path, such as in longitudinal lines along the length of the stent or in circles around the circumference of the stent. In such cases, the apparatus could be controlled to dispense coating only when the dispensing nozzle is adjacent a portion of the stent. The motion may be intermittent or stopped or slowed for dispensing.

FIG. 3 shows an alternative vane type embodiment for a mechanical dispenser portion of a positive displacement coating apparatus in accordance with the invention. That is, instead of the piston type arrangement incorporating syringe plunger 12 moveable within syringe barrel 10, this embodiment uses a vane dispenser 40 comprising a chamber 42 that is swept by a vane member 44. The coating is on the side of the chamber 42 that is attached to dispensing nozzle 14. By computer control, the vane member 44 is caused to pivot about pivot point 46 in the direction of arrow A. This displacement of the vane member in turn causes displacement and dispensing of the coating. As with the piston type arrangement, the mechanical displacement of the vane member 44 directly causes the displacement of the coating.

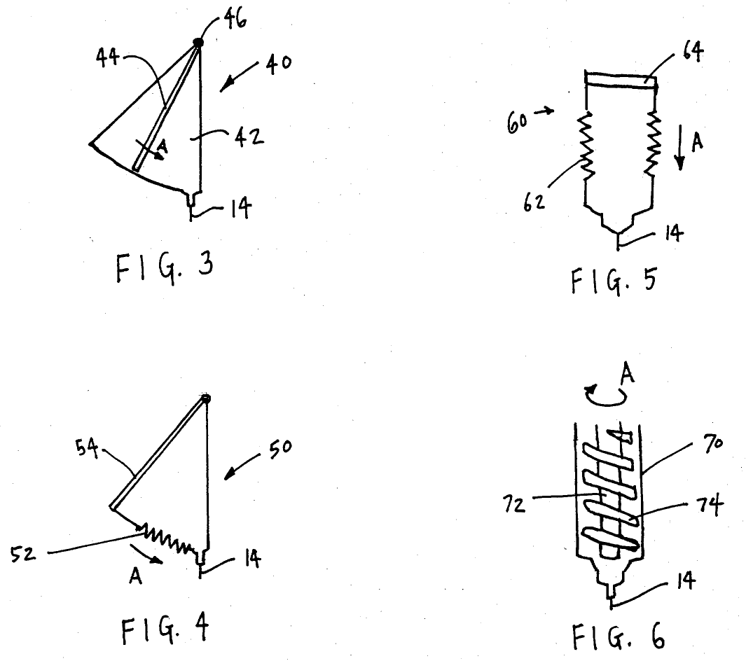

FIG. 4 shows an alternative bellows type embodiment for a mechanical dispenser portion of a positive displacement coating apparatus in accordance with the invention. Here, instead of a piston type or vane type arrangement, this embodiment uses a bellows 50 comprising one or more flexible side walls 52. By computer control, a moveable member 54 is moved in the direction of arrow A. This causes bellows 50 to compress, by the compression of one or more flexible side walls 52. This displacement of the walls of the bellows 50 in turn causes displacement and dispensing of the coating. As with the previously described arrangements, the mechanical displacement of the bellows 50 directly causes the displacement of the coating.

FIG. 5 shows an alternative bladder type embodiment for a mechanical dispenser portion of a positive displacement coating apparatus in accordance with the invention. This embodiment uses a flexible bladder 60 that is similar in some respects to bellows 50. The flexible bladder 60 may comprise a flexible membrane 62. By computer control, a moveable member 64 is moved in the direction of arrow A, causing compression of the flexible membrane 62. This displacement of the flexible membrane 62 in turn causes displacement and dispensing of the coating. As with the previously described arrangements, the mechanical displacement of the flexible membrane 62 directly causes the displacement of the coating.

FIG. 6 shows an alternative screw type embodiment for a mechanical dispenser portion of a positive displacement coating apparatus in accordance with the invention. In this embodiment, a rotatable screw member 72 is located within a tube 70 or other suitable chamber. The screw 72 has threads 74. Fluid entering the top of the tube 70 is forced down the tube and out of the dispensing nozzle 14 by the rotation of the screw member 72. A computer causes the screw to rotate in the direction of the arrow A, and the action of the threads 74 causes positive displacement of the fluid. As with the previously described arrangements, the mechanical displacement of the screw 70 directly causes the displacement of the fluid or coating.

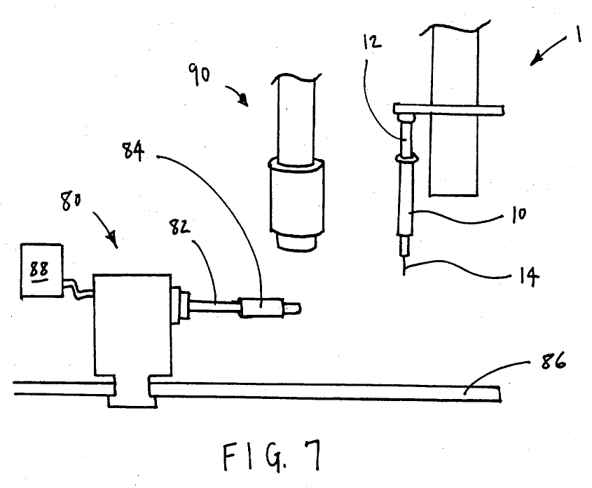

FIG. 7 shows a schematic view of a system for applying a coating to a medical device using a positive displacement coating apparatus in accordance with an embodiment of the invention. The positive displacement coating apparatus 1 is similar to that shown in FIG. 1A, comprising a piston type mechanical dispenser having a syringe barrel 10 and a syringe plunger 12.

Other parts of the system may include a work piece holder 80 and a vision system 90. The work piece holder in this embodiment comprises a spindle 82 on which a stent 84 is shown schematically. The work piece holder 80 is illustrated as being moveable along track 86. A computer processor, shown schematically by box 88, controls the movement along the track 86 as well as the rotation of spindle 82.

The vision system 90 is capable of viewing the position of the stent 84 on the spindle, to determine its precise placement. Persons of ordinary skill in the art will be familiar with vision systems having the capability of performing such a function.

In use, the vision system 90 determines the position of the stent 84 in relation to the parts of the system. To calibrate positioning, the work piece holder 80 may be first moved to a position under the dispensing nozzle 14, so that a drop of coating may be applied to the a portion of the work piece holder, for example the spindle 80. This portion can then be brought under the vision system 90, so that the computer controls of the system know the precise relationship of the parts of the system.

For coating, the work piece holder 80 positions the stent 84 under the dispensing nozzle 14 of the positive displacement coating apparatus 1. Then, by computer control, the work piece holder 80 moves the stent 84 longitudinally and rotationally. Simultaneously, and in coordination, the positive displacement coating apparatus 1 is caused to dispense coating in accordance with the pattern of the stent 84.

As an alternative, the dispensing nozzle 14 can be made to move longitudinally with respect to the stent 84. In this embodiment, the dispensing nozzle 14 may be placed in close proximity to stent 84 and may be moved back and forth along a track so that it may be able to coat the entire external patterned surface of the stent 84. The processor 30 of the positive displacement coating apparatus may instruct it to coat all or only certain portions of the stent 84, but, in any event, it can be programmed so that coating is dispensed only when a portion of the stent is under the dispensing nozzle 14. In other words, as the process is occurring, the positive displacement coating apparatus may force coating onto the surface of the stent 84, while concurrently refraining from forcing coating into spaces between portions of the stent 84. Coating forced into these spaces would simply be wasted or would result in errant deposits of coating elsewhere on the stent 84.

Storage media may be used in communication with the computer processors to store and provide instructions for the processors. Such storage media may be one of numerous types of available storage media including both volatile (i.e. RAM) and non-volatile storage devices (i.e. ROM, CD ROM, EEPROM, Magnetic Media, etc.). The pre-programmed instructions or other retained data may be unique to each medical device to be coated and may account for the unique external pattern and precise dimensions of each medical device to be coated. The storage media may also hold unique instruction sets for many different medical devices or may be provided with a media receptacle such as a disk drive that accommodates different recordable media, each recordable media holding a unique instruction set for a single medical devices or a set of instructions for multiple medical devices.

As mentioned above, a medical device such as stent 84 in this embodiment may be rotated by work piece holder 80 in order to expose different sides of the medical device to the dispensing nozzle 14. Consequently, through the coordinated movement of the medical device and/or the positive displacement coating system, in conjunction with the positive displacement flow of coating, all external portions of the medical device may be exposed to and coated by the dispensing nozzle 14.

In an alternative embodiment, wherein the medical appliance is flat or otherwise linear, the work piece holder 80 may be different than that described above. Here, the work piece holder may provide for movement of the device in both the x and y planes while the dispensing nozzle moves back and forth overhead in order to reach the entire surface of the medical device.

It will be appreciated by persons of ordinary skill in the art that the combined use of the positive displacement coating apparatus, the work piece holder, and the vision system allow the system to perform various operations to locate the parts of the system in relation to each other. For example, the system may be used to locate and orient the medical device by using the vision system to identify the position of an identifiable feature of the medical device. As another example, if the positive displacement coating apparatus is moveable, the system may be used to locate and orient the dispensing nozzle by using the vision system to identify the position of the dispensing nozzle. Alternatively, as previously mentioned, the system may be used to locate and orient the dispensing nozzle by using the vision system to identify the position of a test amount of material ejected by the dispensing nozzle onto a test surface. As another example, the system may be used to monitor disposition of the coating material onto the medical device, by using the vision system to view and/or analyze the medical device after coating.

The positive displacement coating apparatus 1 may be in fluid communication with a suitable coating source. The coating source may contain any one of several possible coatings. These coatings may include paclitaxel, a polymer with a suspended therapeutic, a non-thrombogenic agent, a lubricious material, a non-slippery material, a radiopaque agent, a radioactive agent, and a magnetic signature agent. These coatings may also include: pharmaceutically active compounds, proteins, cells, oligonucleotides, ribozymes, anti-sense oligonucleotides, DNA compacting agents, gene/vector systems (i.e., any vehicle that allows for the uptake and expression of nucleic acids), nucleic acids (including, for example, recombinant nucleic acids; naked DNA, cDNA, RNA; genomic DNA, cDNA or RNA in a non-infectious vector or in a viral vector and which further may have attached peptide targeting sequences; antisense nucleic acid (RNA or DNA); and DNA chimeras which include gene sequences and encoding for ferry proteins such as membrane translocating sequences (“MTS”) and herpes simplex virus-1 (“VP22”)), and viral, liposomes and cationic and anionic polymers and neutral polymers that are selected from a number of types depending on the desired application. Non-limiting examples of virus vectors or vectors derived from viral sources include adenoviral vectors, herpes simplex vectors, papilloma vectors, adeno-associated vectors, retroviral vectors, and the like. Non-limiting examples of biologically active solutes include anti-thrombogenic agents such as heparin, heparin derivatives, urokinase, and PPACK (dextrophenylalanine proline arginine chloromethylketone); antioxidants such as probucol and retinoic acid; angiogenic and anti-angiogenic agents and factors; agents blocking smooth muscle cell proliferation such as rapamycin, angiopeptin, and monoclonal antibodies capable of blocking smooth muscle cell proliferation; anti-inflammatory agents such as serp-1 protein, dexamethasone, prednisolone, corticosterone, budesonide, estrogen, sulfasalazine, acetyl salicylic acid, and mesalamine; calcium entry blockers such as verapamil, diltiazem and nifedipine; antineoplastic antiproliferative/anti-mitotic agents such as paclitaxel, 5-fluorouracil, methotrexate, doxorubicin, daunorubicin, cyclosporine, cisplatin, vinblastine, vincristine, epothilones, endostatin, angiostatin and thymidine kinase inhibitors; antimicrobials such as triclosan, cephalosporins, aminoglycosides, and nitorfurantoin; anesthetic agents such as lidocaine, bupivacaine, and ropivacaine; nitric oxide (NO) donors such as lisidomine, molsidomine, L-arginine, NO-protein adducts, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, Warafin sodium, Dicumarol, aspirin, prostaglandin inhibitors, platelet inhibitors and tick antiplatelet factors; vascular cell growth promotors such as growth factors, growth factor receptor antagonists, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogeneus vascoactive mechanisms; survival genes which protect against cell death, such as anti-apoptotic Bcl-2 family factors and Akt kinase; cladribine; and combinations thereof. Cells may be of human origin (autologous or allogenic) or from an animal source (xenogeneic), genetically engineered if desired. The delivery medium is formulated as needed to maintain cell function and viability. The delivery medium may contain one or more agents to enhance DNA transfection (e.g., poloxamers, cationic polymers, chitosan, etc.), one or more agents to enhance viscosity, and/or one or more agents to enhance cell viability. Any modifications are routinely made by one skilled in the art.

Polynucleotide sequences useful in practice of the invention include DNA or RNA sequences having a therapeutic effect after being taken up by a cell. Examples of therapeutic polynucleotides include anti-sense DNA and RNA; DNA coding for an anti-sense RNA; DNA coding for tRNA or rRNA to replace defective or deficient endogenous molecules; or interfering RNA sequences. The polynucleotides of the invention may also code for therapeutic proteins or polypeptides. A polypeptide is understood to be any translation product of a polynucleotide regardless of size, and whether glycosylated or not. Therapeutic proteins and polypeptides include as a primary example, those proteins or polypeptides that can compensate for defective or deficient species in an animal, or those that act through toxic effects to limit or remove harmful cells from the body. In addition, the polypeptides or proteins that may be injected, or whose DNA may be incorporated, include without limitation, angiogenic factors and other molecules competent to induce angiogenesis, including acidic and basic fibroblast growth factors, vascular endothelial growth factor, hif-1, epidermal growth factor, transforming growth factor α and β, platelet-derived endothelial growth factor, platelet-derived growth factor, tumor necrosis factor α, hepatocyte growth factor and insulin like growth factor; growth factors; cell cycle inhibitors including CDK inhibitors; anti-restenosis agents, including p15, p16, p18, p19, p21, p27, p53, p57, Rb, nFkB and E2F decoys, thymidine kinase (“TK”) and combinations thereof and other agents useful for interfering with cell proliferation, including agents for treating malignancies; and combinations thereof. Still other useful factors, which may be provided as polypeptides or as DNA encoding these polypeptides, include monocyte chemoattractant protein (“MCP-1”), and the family of bone morphogenic proteins (“BMP's”). The known proteins include BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins may be provided as homodimers, heterodimers, or combinations thereof, alone or together with other molecules. Alternatively or, in addition, molecules capable of inducing an upstream or downstream effect of a BMP may be provided. Such molecules include any of the “hedgehog” proteins, or the DNA's encoding them.

A polymeric material may be used in the coating composition as a carrier or matrix for the therapeutic agent. The polymeric material may be either bioabsorbable or biostable. It may be hydrophilic or hydrophobic. The polymeric material may be selected from the group consisting of polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate and blends and copolymers thereof as well as other biodegradable, bioabsorbable and biostable polymers and copolymers. Coatings from polymer dispersions such as polyurethane dispersions (BAYHDROL®, etc.) and acrylic latex dispersions may also be used. The polymer may be a protein polymer, fibrin, collage and derivatives thereof, polysaccharides such as celluloses, starches, dextrans, alginates and derivatives of these polysaccharides, an extracellular matrix component, hyaluronic acid, or another biologic agent or a suitable mixture of any of these, for example. One example of a polymer that may be used is polyacrylic acid, available as HYDROPLUS® (Boston Scientific Corporation, Natick, Mass.), and described in U.S. Pat. No. 5,091,205, the disclosure of which is hereby incorporated herein by reference. U.S. Pat. No. 5,091,205 describes medical devices coated with one or more polyisocyanates such that the devices become instantly lubricious when exposed to body fluids. The polymer may be a copolymer, for example, of polylactic acid and polycaprolactone.

Another alternative coating material is any conductive material, which may be coated on the medical appliance to provide electrical conductivity for either power or signal functions to different parts of the medical appliance. For instance, an electrically conductive stripe may be applied to a catheter to enable a source of power at a proximal end of the catheter to provide power to a remote application at a distal end of the catheter. Additionally, the positive displacement coating apparatus may be utilized to coat a previously applied conductive material with an insulating material to thereby electrically isolate the conductive material.

A positive displacement coating apparatus may enable coating with more viscous materials than alternative methods because it may have a larger orifice and nozzle through which the coating fluids travel. Coating materials may become viscous due to a high solids content, which may be due to a higher concentration of therapeutic. A higher concentration of therapeutic may be preferable from a clinical standpoint in that it may make the medical appliance more effective. Additionally, coatings having high concentrations of therapeutic (and therefore high viscosity) may require fewer coating steps, and therefore require less time to produce. Therefore, higher drug loads may be applied to the medical appliance with fewer coats which may be applied in less time.

In addition, because the positive displacement coating apparatus controls the coating flow by computer control of a mechanical dispensing mechanism, the amount of coating being dispensed may be determined and controlled precisely.

The positive displacement coating apparatus in this embodiment is preferably programmed to coat in a precise manner, allowing coating to be applied in a complex pattern, matching the complex pattern of the medical device. It may also be preferred that the stream of coating forced from the dispensing nozzle be small in relation to the target area of the medical device to allow for a high degree of precision in coating the target. Precision coating of the medical device enables economical use of coating materials.

In an alternative embodiment, rather than having the coating material deposited in one coat or layer around the entire device, the positive displacement coating apparatus may coat the medical device with different layers of different thicknesses in different regions of the device as may be desirable for the subsequent use of the device. In doing so, different concentrations of therapeutic may be deposited in different regions of the medical device. Additionally or alternatively, the positive displacement coating apparatus may be used to apply different compositions of coatings to different areas of a device, to apply compositions in different thicknesses to different areas of the device, and/or to apply compositions in layers to all or parts of the device. Differences in layers, thicknesses and/or compositions may be used, for example, to control release of therapeutic over time.

The coatings that may be applied by a positive displacement coating apparatus may also include: lubricious coatings to reduce the stress exerted on the stent during the stent's deployment; radiopaque coatings for identifying the location of stents after implantation using traditional radiography techniques; radioactive agents that are useful in preventing tissue regrowth in and around implanted stents; and magnetic coatings that enable identification of the location of the implanted stent using Magnetic Resonance Imaging (MRI) techniques. These magnetic coatings may be obtained using ferritic powders or paramagnetic powders such as Gadolinium or Disprosium.

Another useful application of this precise coating method may be to convey information, or an identification code on the appliance itself. This information or code may then be used to identify the source of the medical appliance and other history related to it for tracking purposes. Once implanted, the code, which may be a bar code, could be read though radiography, MRI or any other suitable invasive or non-invasive procedure.

The mechanism for holding the medical device may take any of a number of suitable forms. For example, a mechanism may be used comprising a notch system and support cylinders. The mechanism may also include means for measuring the weight of the medical device (e.g, balance/load cell), to determine the amount of coating that has been applied.

While several embodiments have been discussed, others, within the invention's spirit and scope, are also plausible. For example, while one dispensing nozzle is described in each of the above embodiments, more than one dispensing nozzle may also be employed. In this alternative embodiment, the multiple dispensing nozzles may work synchronously and asynchronously and may be ganged together to coat several medical devices simultaneously. As another example, valves such as valve 18 may be incorporated with any of the various types of described dispensers. Other variations are within the scope of the invention, as defined by the appended claims.

What is claimed is:

1. A positive displacement coating apparatus for applying a coating to a medical device having an accessible surface, the apparatus comprising:
a computer processor;
a motor; and
a mechanical dispensing mechanism comprising a chamber and means for displacing coating within the chamber;
wherein the computer processor controls movement of the motor, and wherein the motor is mechanically linked to the means for displacing coating within the chamber, so that movement of the motor causes movement of the means for positively displacing coating within the chamber and consequently causes movement of coating out of the chamber.

2. The positive displacement coating apparatus of claim 1, wherein the chamber is a syringe barrel and the means for displacing coating within the chamber is a syringe plunger.

3. The positive displacement coating apparatus of claim 1, wherein the means for displacing coating within the chamber is a moveable vane member.

4. The positive displacement coating apparatus of claim 1, wherein the chamber is a bellows and the means for displacing coating within the chamber is a member that compresses the bellows.

5. The positive displacement coating apparatus of claim 1, wherein the chamber is a compressible bladder defined by a flexible membrane and the means for displacing coating within the chamber is a member that compresses the flexible membrane.

6. The positive displacement coating apparatus of claim 1, wherein the means for displacing coating within the chamber is a rotatable screw propeller.

7. A system for applying a coating to a medical device having an accessible surface using a positive displacement coating apparatus, the system comprising:
a work piece holder for holding the medical device to be coated;
a vision system; and
a positive displacement coating apparatus comprising:
a computer processor;
a motor; and
a mechanical dispensing mechanism comprising a chamber and means for displacing coating within the chamber;
wherein the computer processor controls movement of the motor, and wherein the motor is mechanically linked to the means for displacing coating within the chamber, so that movement of the motor causes movement of the means for positively displacing coating within the chamber and consequently causes movement of coating out of the chamber.

8. The system for applying a coating to a medical device of claim 7, wherein the chamber is a syringe barrel and the means for displacing coating within the chamber is a syringe plunger.

9. The system for applying a coating to a medical device of claim 7, wherein the means for displacing coating within the chamber is a moveable vane member.

10. The system for applying a coating to a medical device of claim 7, wherein the chamber is a bellows and the means for displacing coating within the chamber is a member that compresses the bellows.

11. The system for applying a coating to a medical device of claim 7, wherein the chamber is a compressible bladder defined by a flexible membrane and the means for displacing coating within the chamber is a member that compresses the flexible membrane.

12. The system for applying a coating to a medical device of claim 7, wherein the means for displacing coating within the chamber is a rotatable screw propeller.

13. The system for applying a coating to a medical device of claim 7, wherein the work piece holder is adapted to spin the medical device about a longitudinal axis of the medical device.

14. The system for applying a coating to a medical device of claim 7, wherein the system is adapted to perform at least one of the following functions:
locate and orient the medical device by identifying the position of an identifiable feature of the medical device;
locate and orient a dispensing nozzle of the positive displacement coating apparatus by identifying at least one of a position of the dispensing nozzle and a test amount of material ejected by the dispensing nozzle onto a test surface; and
monitor disposition of the coating material onto the accessible surface of the medical device.

15. The system for applying a coating to a medical device of claim 7, wherein the computer processor includes:
a memory, the memory storing data that represents a configuration of the accessible surface of the medical device; and
a control unit, the control unit generating command signals that instruct the positive displacement coating apparatus to force coating onto the accessible surface of the medical device in a pattern that correlates with the accessible surface of the medical device being held by the work piece holder.

16. A method for applying a coating onto a medical device having an accessible surface, the method comprising:
holding the medical device and providing direct access to the accessible surface of the medical device; and
receiving command signals at a positive displacement coating apparatus, the command signals including instructions to force coating onto the accessible surface of the medical device in a pattern that correlates with the accessible surface of the medical device; and
mechanically displacing a moveable member of the positive displacement coating apparatus, wherein the mechanical displacement of the moveable member causes displacement of a coating onto the accessible surface of the medical device.

17. The method of claim 16 further comprising spinning the medical device about a longitudinal axis.

18. The method of claim 17 further comprising at least one of the following steps:
locating and orienting the medical device by positioning an identifiable feature of the medical appliance;
locating and orienting a dispensing nozzle of the positive displacement coating apparatus by at least one of positioning the dispensing nozzle and positioning a test amount of material ejected by the dispensing nozzle onto a test surface; and
monitoring disposition of the coating material onto the accessible surface of the medical device.

19. The method of claim 17 further comprising:
storing data that represents the configuration of the accessible surface of the medical device; and
generating command signals that instruct the positive displacement coating apparatus to force coating onto the accessible surface of the medical device in a pattern that correlates with the accessible surface of the medical device.
